(19) United States
(12) Patent Application Publication
(10) Pub. No.: US 2020/0246351 A1
(43) Pub. Date: Aug. 6, 2020

(54) COMBINATIONS AND USES AND TREATMENTS THEREOF

(71) Applicant: ViiV Healthcare Company, Wilmington, DE (US)

(72) Inventors: Mark I. COCKETT, Connecticut, PA (US); Eugene L. STEWART, Collegeville, PA (US); Mark Richard UNDERWOOD, Research Triangle Park, NC (US); Cindy L. VAVRO, Research Triangle Park, NC (US)

(21) Appl. No.: 16/636,477

(22) PCT Filed: Aug. 2, 2018
(86) PCT No.: PCT/IB18/55828

§ 371 Date: Feb. 4, 2020

Related U.S. Application Data
(60) Provisional application No. 62/542,924, filed on Aug. 9, 2017.

(51) Int. Cl. A61K 31/553; A61K 31/506; A61P 31/18

(52) U.S. Cl. CPC A61K 31/553; A61P 31/18; A61K 31/506

(57) ABSTRACT

Methods for treating or preventing HIV in a patient using a combination of bictegravir and lamivudine and optionally with other anti-HIV agents are disclosed, as well as compositions containing such compounds.

### FIELD OF THE INVENTION

Methods for treating or preventing HIV in a patient using a combination of bictegravir and lamivudine as well as compositions comprising such compounds.

### BACKGROUND OF THE INVENTION

Human immunodeficiency virus infection and related diseases are a major public health problem worldwide. Human immunodeficiency virus type 1 (HIV-1) encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, et al. N. Engl. J. Med. (1998) 338:853-860; Richman, D. D. Nature (2001) 410:995-1001).

A goal of antiretroviral therapy is to achieve viral suppression in the HIV infected patient. Treatment guidelines published by the United States Department of Health and Human Services provide that achievement of viral suppression requires the use of combination therapies, i.e., several drugs from at least two or more drug classes. (Panel on Antiretroviral Guidelines for Adults and Adolescents. Guidelines for the use of antiretroviral agents in HIV-1-infected adults and adolescents. Department of Health and Human Services. Available at http://aidsinfo.nih.gov/ContentFiles/AdultandAdolescentGL.pdf. Section accessed Mar. 14, 2013.) In addition, decisions regarding the treatment of HIV infected patients are complicated when the patient requires treatment for other medical conditions (Id. at E-12). Because the standard of care requires the use of multiple different drugs to suppress HIV, as well as to treat other conditions the patient may be experiencing, the potential for drug interaction is a criterion for selection of a drug regimen. As such, there is a need for antiretroviral therapies having a decreased potential for drug interactions and with even more therapeutic potencies.

US9216996 describes a class of HIV integrase of substituted 2,3,4,5,7,9,13,13a-octahydropyrido[1′,2′:4,5]pyrazino[2,1-b ][1,3] oxazepines. Among them is an agent called bictegravir (otherwise known as GS-9883). Bictegravir has structure Formula I,

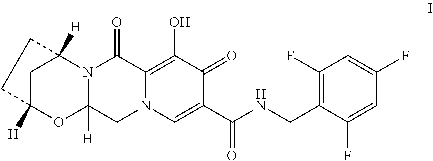 [SMILES: O=C(NCc1c(F)cc(F)cc1F)c1cn2c(c(O)c1=O)C(=O)N1C(C2)O[C@@H]2CC[C@H]1C2]

and is undergoing clinical trials in combination with emtricitabine (FTC), and tenofovir alafenamide (TAF).

Lamivudine (3TC) is a nucleoside reverse transcriptase inhibitor (NRTI) of structural Formula II.

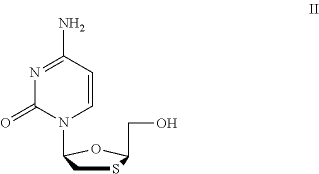

Although different combinations of antiretroviral drugs have been developed for the treatment of HIV, a need still exists for further HIV treatment regimens for even more potencies or ease of use.

A standard course of care for a patient infected with HIV is to treat them with a combination of three or more antiviral agents. Frequently, this treatment uses at least one antiretroviral agents targeting HIV reverse transcriptase (a “backbone”) and/or one or more agents active against one or more different HIV targets, such as an HIV protease inhibitor, an HIV non-nucleoside or non-nucleotide inhibitor of reverse transcriptase, an HIV nucleoside or nucleotide inhibitor of reverse transcriptase, an HIV integrase inhibitor, an HIV non-catalytic site (or allosteric) integrase inhibitor, or a combination thereof. For certain patients infected with HIV or diagnosed with AIDS, there is an unmet medical need to treat them with fewer antiviral agents.

While ART has led to substantial increases in life expectancy and quality of life for HIV-infected persons, HIV infection requires lifelong treatment. This means that as HIV-infected individuals achieve life expectancies near those of persons without HIV, HIV-infected individuals are likewise starting to receive treatment for non-HIV, common conditions such as diabetes, cardiovascular disease, arthritis, osteoporosis, or other age-associated conditions and diseases. (Zhou et al., Total Daily Pill Burden in HIV-Infected Patients in the Southern United States, 2014 AIDS PATIENT CARE and STDs 28(6): 311-317.) This increased drug burden (of HIV patients also now taking medications for HIV-unrelated indications) raises risks of drug-drug interactions and overlapping toxicities, not to mention it increases the patient's healthcare costs and dosing hassle. (Zhou et al., AIDS PATIENT CARE and STDs 28(6): 311-317.) Further, increasing medication complexity may affect treatment adherence and virologic suppression. (Zhou et al., AIDS PATIENT CARE and STDs 28(6): 311-317.)

Fewer drugs in HIV infected patients are also desired for those that are likely to tolerate two drugs rather than more such as aging patients, those with advanced HIV infections or other diseases, or to avoid drug-drug interactions, and to limit side effects among patients. Thus, there is a need for new treatment regimens which suppress viral load in humans having HIV where the treatment regimen comprises only two antiviral agents.

Additionally, an issue associated with administration of HIV medications, including both bictegravir and lamivudine, is patient compliance. Because all HIV drugs must be taken as part of a combination regimen, there must be better ways to ensure patient compliance in taking medication as prescribed. If there are too many pills to swallow, at too many time intervals, then dosing becomes inconvenient and complicated, and patient compliance with the treatment regimen is less likely.

Thus, what is needed are new, easily administered, combination formulations containing potent antiretroviral drugs which are useful in the treatment of HIV infection. These new two drug formulations should be convenient and easy to administer, as well as showing good physical stability and low degradant levels.

In particular, stable, easily administered fixed dose combinations (FDCs) of bictegravir and lamuvidine is desired.

### SUMMARY OF THE INVENTION

One embodiment of the invention provides a method for treating or preventing HIV infection in a human patient comprising administering to the patient a pharmaceutically effective amount of bictegravir, or a pharmaceutical composition thereof; and an effective amount of lamivudine, or a pharmaceutical composition thereof.

In another embodiment, bictegravir and lamivudine are co-administered in separate dosage forms.

In another embodiment, bictegravir and lamivudine are co-administered in a single dosage form.

In yet another embodiment, combinations comprising bictegravir, or a pharmaceutically acceptable salt thereof, and lamivudine, or a pharmaceutically acceptable salt thereof, are provided.

In yet another embodiment, combinations consisting essentially of bictegravir, or a pharmaceutically acceptable salt thereof; and lamivudine, or a pharmaceutically acceptable salt thereof, are provided.

In yet another embodiment, combinations consisting of bictegravir, or a pharmaceutically acceptable salt thereof; and lamivudine, or a pharmaceutically acceptable salt thereof; and one or more pharmaceutically acceptable carriers, diluents or excipients are provided.

In an additional embodiment, this invention provides a combination of bictegravir, or a pharmaceutically acceptable salt thereof, and lamivudine, or a pharmaceutically acceptable salt thereof, for use in medical therapy.

One embodiment of the invention provides a method for treating HIV infection in a human patient who are virologically suppressed (HIV-1 RNA<50c/mL) administering to the patient a pharmaceutical composition comprising bictegravir, or a pharmaceutically acceptable salt thereof; and lamivudine, or a pharmaceutically acceptable salt thereof. In another embodiment, the present invention provides a method of maintaining HIV-1 RNA <50 c/mL by administering to the patient a pharmaceutical composition comprising bictegravir, or a pharmaceutically acceptable salt thereof; and lamivudine, or a pharmaceutically acceptable salt thereof. In another embodiment, the maintenance is achieved by switching from an antiretroviral regimen including 2 nucleoside reverse transcriptase inhibitors plus a third agent. Yet in another embodiment, HIV-1 RNA<50 c/mL is maintained even at 48 weeks. In one embodiment of the invention, the pharmaceutical composition consists of two active ingredients of bictegravir and lamivudine, and one or more pharmaceutically acceptable excipients, diluents or carriers.

In another embodiment, the patient has received three or more antiviral agents prior to receiving the pharmaceutical composition of bictegravir and lamuvidine. For example, a patient might have received antiretroviral regimen (two nucleoside reverse transcriptase inhibitors [NRTIs] + a third agent). The third agent could be either integrase inhibitor (INI), non-nucleoside reverse transcriptase inhibitor (NNRTI), or protease inhibitor (PI). In one embodiment, the patient has received an antiretroviral regimen comprising bictegravir, tenofovir or tenofovir prodrug, such as tenofovir disoproxil fumarate (TDF) or TAF (including hemi-fumarate and mono-fumarate), or emtricitabine prior to receiving the pharmaceutical composition of bictegravir and lamuvidine. In another embodiment, the patient has shown resistance to either bictegravir, tenofovir, and/or emtricitabine prior to receiving the pharmaceutical composition of bictegravir and lamuvidine. In a more preferred embodiment, a patient is switched to a pharmaceutical composition of bictegravir and lamuvidine from a regimen comprising bictegravir (e.g. a combination of bictegravir, TAF, and emtricitabine).

In one embodiment of the invention, methods are provided of treating or preventing HIV-1 or HIV-2 (in particular for HIV-1) in a virologically suppressed patient in need thereof comprising switching the patient from an antiretroviral treatment regimen comprising at least three antiretroviral agents to a treatment regimen comprising only two antiretroviral agents.

In another embodiment of the invention is a method of treating HIV-1 or HIV-2 (in particular for HIV-1) in a virologically suppressed patient in need thereof comprising switching the patient from an antiretroviral treatment regimen comprising at least three antiretroviral agents to a treatment regimen comprising only two antiretroviral agents wherein the first antiviral agent is a therapeutically effective amount of a compound of formula I, or with an additional pharmaceutically acceptable salt thereof; and the second antiviral agent is a therapeutically effective amount of a compound of Formula II, or with an additional pharmaceutically acceptable salt thereof. Thus, in one embodiment, methods are provided of treating HIV-1 or HIV-2 (in particular for HIV-1) in a virologically suppressed patient in need thereof comprising switching the patient from an antiretroviral treatment regimen comprising at least three antiretroviral agents to a treatment regimen comprising only two antiretroviral agents essentially consisting of bictegravir sodium (or with other suitable cation) equivalent to 50 mg bictegravir free acid and 150mg or 300mg of lamuvidine and at least one inactive ingredient.

In another embodiment of the invention is a method of treating or preventing HIV-1 or HIV-2 (in particular for HIV-1) in a virologically suppressed patient in need thereof comprising switching the patient from an antiretroviral treatment regimen comprising at least three antiretroviral agents to a treatment regimen comprising only two antiretroviral agents essentially consisting of about 50 mg. per dose of a compound of Formula I, or with an additional pharmaceutically acceptable salt thereof, and about 150 or 300 mg. per dose of a compound of Formula II, or with an additional pharmaceutically acceptable salt thereof. In another embodiment, a method or composition comprises between about 1 mg. and 200 mg. of a compound of Formula I, or with an additional pharmaceutically acceptable salt thereof, and between about 1 mg. and 300 mg. of a compound of Formula II, or with an additional pharmaceutically acceptable salt thereof. In another embodiment, a method or composition comprises between 10 mg. and 100 mg. of a compound of Formula I, or with an additional pharmaceutically acceptable salt thereof, and between 10 mg. and 300 mg. of a compound of Formula II, or with an additional pharmaceutically acceptable salt thereof.

Yet another embodiment comprises such equivalents of 10 mg., 20 mg, 25 mg., 30, mg, 35 mg, 40 mg, 45 mg, 50 mg., 75 mg., 100 mg. of a compound of Formula I, or with an additional pharmaceutically acceptable salt thereof, and 10 mg., 20 mg, 25 mg., 30 mg, 35 mg, 40 mg, 45 mg, 50 mg., 75 mg., 100 mg., 150mg, 300mg. of a compound of Formula II, or with an additional pharmaceutically acceptable salt thereof. In a further embodiment, a tablet, or other composition may comprise with an additional pharmaceutically acceptable form of bictegravir equivalent to 50 mg. bictegravir free acid and comprise with an additional pharmaceutically acceptable form of 150 mg or 300 mg of lamuvidine. Provided as an embodiment for any dose range of the invention is each integer dose amount between each end number of a dose range. For example, a dose range from 15 mg. to 50 mg. would also include 16 mg., 17 mg., and so on up to 49 mg (including all decimal points, fractions, and integers, in between each value). A value of about 50 mg. would include values greater than 45 mg. and also values less than 55 mg. Other therapeutically effective doses of bictegravir and lamuvidine can be determined or optimized using known pharmaceutical or clinical practices.

In one embodiment, the antiviral regimens may each comprise any number of steps or undergo any number of manipulations and the compositions used in each regimen may comprise any number of components, such as excipients or biologically active compounds (e.g., non-antiviral pharmaceutical compounds); however, with regard to the number of antiviral agents in the first antiviral regimen and its composition that number is limited to three or more antiviral agents, but no fewer, and with regard to the number of antiviral agents in the second antiviral regimen and its composition that number is limited to two antiviral agents, no more nor fewer.

In one embodiment, a treatment regimen is provided that comprises switching from an antiviral treatment regimen comprising at least three antiviral agents comprising of one or more antiviral compounds selected from the group of: an HIV protease inhibitor, an HIV non-nucleoside or non-nucleotide inhibitor of reverse transcriptase, an HIV nucleoside or nucleotide inhibitor of reverse transcriptase, an HIV integrase inhibitor, MK8591 (EFdA), an HIV non-catalytic site (or allosteric) integrase inhibitor, an HIV entry inhibitor (e.g., a CCRS inhibitor, a gp41 inhibitor (i.e., a fusion inhibitor) or a CD4 attachment inhibitor (e.g., combinectin), a CXCR4 inhibitor, a gp120 inhibitor, a G6PD or an NADH-oxidase inhibitor, an HIV vaccine, a latency reversing agent (e.g., a histone deacetylase inhibitor, a proteasome inhibitor, a protein kinase C (PKC) activator, or a BRD4 inhibitor), a compound that targets HIV capsid (a “capsid inhibitor”; e.g., a capsid polymerization inhibitor or a capsid disrupting compound, an HIV nucleocapsid p7 (NCp7) inhibitor, an HIV p24 capsid protein inhibitor), a pharmacokinetic enhancer, an immune-based therapy (e.g., a Pd-1 modulator, a Pd-L1 modulator, a CTLA4 modulator, an ICOS modulator, an OX40 modulator, or the like, a toll-like receptor modulator, an IL-15 agonist, an anti-HIV antibody, a bispecific antibody or an “antibody-like” therapeutic protein (e.g., a DART, a DUOBODY, a BITE, an XmAb, a TandAb, a Fab derivative) including those targeting a HIV gp120 or gp41, combination drug for HIV, an HIV p 17 matrix protein inhibitor, an IL-13 antagonist, a peptidylprolyl cis-trans isomerase A modulator, a protein disulfide isomerase inhibitor, a complement C5a receptor antagonist, a DNA methyltransferase inhibitor, an HIV vif gene modulator, a Vif dimerization antagonist, an HIV-1 viral infectivity factor inhibitor, a TAT protein inhibitor, an HIV-1 Nef modulator, an Hck tyrosine kinase modulator, a mixed lineage kinase-3 (MLK-3) inhibitor, an HIV-1 splicing inhibitor, aRev protein inhibitor, an integrin antagonist, a nucleoprotein inhibitor, a splicing factor modulator, a COMM domain containing protein 1 modulator, an HIV ribonuclease H inhibitor, a retrorocyclin modulator, a CDK-9 inhibitor, a dendritic ICAM-3 grabbing nonintegrin 1 inhibitor, an HIV GAG protein inhibitor, an HIV

POL protein inhibitor, acomplement Factor H modulator, a ubiquitin ligase inhibitor, a deoxycytidine kinase inhibitor, a cyclin dependent kinase inhibitor, a proprotein convertase PC9 stimulator, an ATP-dependent RNA helicase DDX3X inhibitor, a reverse transcriptase priming complex inhibitor, an HIV gene therapy, a PI3K inhibitor, a compound, such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US 20140221378 (Japan Tobacco), WO 2010/130034 (Boehringer Ingelheim), WO 2013/159064 (Gilead Sciences), WO 2012/145728 (Gilead Sciences), W02012/003497 (Gilead Sciences), WO2014/100323 (Gilead Sciences), WO2012/145728 (Gilead Sciences), WO 2013/159064 (Gilead Sciences) and WO 2012/003498 (Gilead Sciences) and WO 2013/006792 (Pharma Resources), and other drugs for treating HIV.

In one embodiment, a treatment regimen is provided that comprises switching from an antiviral treatment regimen comprising at least three antiviral agents. In another embodiment, a treatment regimen is provided that comprises switching from an antiviral treatment regimen comprising bictegravir, tenofovir or tenofovir prodrug, such as tenofovir disoproxil fumarate (TDF) or TAF (including hemi-fumarate and mono-fumarate), and/or emtricitabine.

In another embodiment, the regimen comprises switching from using a composition of the invention to using a composition comprising a combination comprising one or more of the aforementioned antiviral compounds. Another embodiment provides a method comprising an antiretroviral regimen comprising two NRTIs and one or more of an antiretroviral agent selected from the group consisting of an INI, an NNRTI, or a PI.

In another embodiment of the method the human or patient is virologically suppressed. By way of example a patient is considered virologically suppressed if the patient has an HIV copy number of between 0 and 200 copies per mL, less than 20 copies per mL, 50 copies per mL, 100 copies per mL, and/or 200 copies per mL. Provided also as an embodiment for any copy number of the invention are each integer copy number between each end number of a copy number range. For example, a copy number range from 20 copies per mL to 50 copies per mL would also include 21, 22, 23 up to 49 copies per mL.

An embodiment of the invention provides a composition of the invention administered to a patient infected with wild-type HIV-1 or HIV-2 (in particular for HIV-1), an HIV Glade B virus, an HIV of M Glade A, B, C, D, E, F, G, or H or an HIV group 0 virus, or mutants thereof.

An embodiment of a regimen of the invention provides administering a composition of the invention to a patient infected with a certain mutant HIV-1 virus or HIV-2 (in particular for HIV-1) virus, such as a mutant virus comprising a single amino acid substitution or two or more substitutions. Certain of such regimens provide administering a composition of the invention to a patient infected with an INSTI substitution mutant, such as a raltegravir-resistant mutant, or an elvitegravir-resistant mutant.

HIV mutations showing NRTI resistance is well documented. Examples of HIV mutations which show resistance to TAF (tenofovir alafenamide fumarate) (TAF has the same resistance profile tenovofir and tenofovir disoproxil) and FTC (emtricitabine) are published, such as, in Characterization of HIV-1 Resistance to Tenofovir Alafenamide In vitro, Antimicrobial Agents and Chemotherapy, vN. A. Margot et al., Volume 59 Number 10(2015). Also is published online at https://hivdb.stanford.edu/dr-summary/resistance-notes/NRTI/.

Also provided is an embodiment that is a regimen of the invention or composition of the invention administered to or used to treat an anti-retroviral treatment (ART) experienced patient. A certain embodiment provides that this patient is also virologically suppressed.

Regimens of the invention and compositions of the invention are used to treat patients with infected wild type or mutant HIV or virus comprising an HIV integrase homolog. In another embodiment, the invention provides a method to administer a composition of the invention to a treatment-experienced patient, such as a patient that is virologically-suppressed.

In another embodiment, the patient has HIV-1 or HIV-2 (in particular for HIV-1) RNA less than 50 copies per mL prior to switching from an antiretroviral treatment regimen comprising at least three antiretroviral agents to a treatment regimen comprising only two antiretroviral agents. In another embodiment, the patient has HIV-1 or HIV-2 (in particular for HIV-1) RNA less than 50 copies per mL prior to switching from an antiretroviral treatment regimen comprising at least three antiretroviral agents to a treatment regimen comprising a compound of Formula I or with an additional pharmaceutically acceptable salt thereof; and a compound of Formula II, or with an additional pharmaceutically acceptable salt thereof. In another embodiment, the switching to a treatment regimen comprising a compound of Formula I or with an additional pharmaceutically acceptable salt thereof; and a compound of Formula II, or with an additional pharmaceutically acceptable salt thereof occurs after at least 6 months of virologically suppression (HIV-1 RNA less than 50 copies per mL) with no history of treatment failure and no known substitutions associated resistance with the compound of Formula I or compound of Formula II.

In another embodiment, the present invention provides a method of maintaining HIV-1 or HIV-2 (in particular for HIV-1) RNA less than 50 copies per mL by administering to the patient a pharmaceutical composition of the invention comprising a compound of Formula I, or with an additional pharmaceutically acceptable salt thereof; and a compound of Formula II, or with an additional pharmaceutically acceptable salt thereof. In another embodiment, the pharmaceutical composition comprises a compound of Formula I, or with an additional pharmaceutically acceptable salt thereof; and a compound of Formula II, or with an additional pharmaceutically acceptable salt thereof, and one or more pharmaceutically acceptable excipients, diluents or carriers. In an additional embodiment, HIV-1 or HIV-2 (in particular for HIV-1) RNA less than 50 copies per mL is maintained at 48 weeks after switching treatment regimens from a three or more antiretroviral regimen to treatment regimen consisting of a compound of Formula I, or with an additional pharmaceutically acceptable salt thereof, and a compound of Formula II, or with an additional pharmaceutically acceptable salt thereof, and at least one excipient, diluent or carrier.

In yet another embodiment, bictegravir and lamivudine are orally co-administered.

In yet another embodiment, bictegravir is administered parenterally and lamivudine is administered orally.

In one embodiment, pharmaceutical compositions comprising bictegravir and lamivudine, and a pharmaceutically acceptable carrier, diluent or excipient are provided.

In one embodiment, a single dosage form containing bictegravir and lamivudine is administered 4 hours before or 6 hours after taking antiacids, containing aluminum, magnesium hydroxide, and/or calcium carbonate. In one embodiment, the single dosage form containing bictegravir and lamivudine is administered 4 hours before or 6 hours after taking oral non-antiacid supplements containing iron or calcium. Alternatively, the single dosage form (containing bictegravir and lamivudine) and supplements containing iron or calcium is taken with food. In one embodiment, the co-administration, separately or in a single dosage form, of bictegravir and lamivudine is administered to patients with HIV strains which are resistant to NRTI other than lamivudine, in particular, to emtricitabine and/or TAF. In another embodiment, the co-administration, separately or in a single dosage form, of bictegravir and lamivudine is switching from NRTI other than lamivudine to which HIV strain is showing resistance, in particular to emtricitabine and/or TAF.

Another embodiment provides a method for preventing an HIV infection or AIDS, comprising administering to a human a therapeutically effective amount of a compound of Formula I, or with an additional pharmaceutically acceptable salt thereof, and a therapeutically effective amount of a compound of Formula II, or with an additional pharmaceutically acceptable salt thereof, to a patient who is at risk of acquiring HIV infection. For example, methods may be prophylactic for an intravenous drug abuser, a person who contacts or has a likelihood of contacting bodily fluid from an HIV-infected individual, or a person who engages or may engage in a sexual or other activity associated with a risk of acquiring an HIV infection.

An embodiment of the invention provides a therapeutically effective regimen of the invention or a therapeutically effective composition of the invention. Any embodiment of the invention that comprises or relates to a patient also comprises or relates to a human. Any composition of the invention can be administered to a human. Any regimen of the invention can be used on a human, for example to treat a human, such as a human infected with HIV.

In one embodiment of this invention, combinations are provided of only two antiviral agents, those being a compound of Formula I, or a pharmaceutically acceptable salt thereof, and a compound of Formula II, or a pharmaceutically acceptable salt thereof, for use in treating HIV-1 or HIV-2 (in particular for HIV-1) in a virologically suppressed patient in need thereof comprising switching the patient from an antiretroviral treatment regimen comprising at least three antiretroviral agents to a treatment regimen comprising only two antiretroviral agents. In some embodiments, the combination further comprises at least one pharmaceutically acceptable excipient, diluent, and/or carrier. Combinations of the present invention can comprise a first antiretroviral agent being bictegravir, and the second antiretroviral agent being lamuvidine. Uses of these combinations include treating or preventing HIV-1 or HIV-2 (in particular for HIV-1) in a virologically suppressed patient in need thereof. The disclosed methods of treatment and uses can be used in connection with the combinations.

In certain embodiments, a method for treating or preventing an HIV infection in a human patient comprising administering to the human a therapeutically effective amount of bictegravir and lamivudine, or a pharmaceutical composition thereof, in combination with a therapeutically effective amount of one or more (e.g., one, two, three, one or two, or one to three) additional therapeutic agents.

In one embodiment, pharmaceutical compositions comprising bictegravir and lamivudine, in combination with one or more (e.g., one, two, three, one or two, or one to three) additional therapeutic agents, and a pharmaceutically acceptable carrier, diluent or excipient are provided.

In yet another embodiment, bictegravir is administered orally to assess the safety and tolerability, and if no or low issue in safety and tolerability is found (called “oral-lead method”), then bictegravir is administered intramuscularly or subcutaneously.

In one embodiment, kits comprising bictegravir and lamivudine, or a pharmaceutical composition thereof, in combination with one or more (e.g., one, two, three, one or two, or one to three) additional therapeutic agents are provided. In a further embodiment, kits comprising bictegravir and lamivudine, or a pharmaceutical composition thereof, in oral and/or parenteral dosage forms are provided. Certain such kits comprise bictegravir in a syringe dosage or tablet dosage form, and lamivudine in a syringe dosage or tablet dosage form.

In one embodiment, the above-described compositions, kits or combinations for use in medical therapy are provided. In another embodiment, the above-described compositions, kits or combinations for use in any of the above-described methods are provided.

In the above embodiments, the additional therapeutic agent may be an anti-HIV agent. For example, in some embodiments, the additional therapeutic agent is chosen from: HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, HIV vaccines, HIV maturation inhibitors, latency reversing agents (e.g., histone deacetylase inhibitors, proteasome inhibitors, protein kinase C (PKC) activators, and BRD4 inhibitors), compounds that target the HIV capsid (“capsid inhibitors”; e.g., capsid polymerization inhibitors or capsid disrupting compounds, HIV nucleocapsid p7 (NCp7) inhibitors, HIV p24 capsid protein inhibitors), pharmacokinetic enhancers, immune-based therapies (e.g., Pd-1 modulators, Pd-Ll modulators, CTLA4 modulators, toll like receptors modulators, IL-15 agonists, HIV antibodies, bispecific antibodies and “antibody-like” therapeutic proteins (e.g., DARTs®, DUOBODIES ®, BITES®, XmAbs®, TandAbs®, Fab derivatives) including those targeting HIV gp120 or gp41, combination drugs for HIV, HIV p 17 matrix protein inhibitors, IL-13 antagonists, Peptidylprolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV Ribo-nuclease H inhibitors, retrorocyclin modulators, CDK-9 inhibitors, dendritic ICAM-3 grabbing nonintegrin 1 inhibitors, HIV GAG protein inhibitors, HIV POL protein inhibitors, complement Factor H modulators, ubiquitin ligase inhibitors, deoxycytidine kinase inhibitors, cyclin dependent kinase inhibitors proprotein convertase PC9 stimulators, ATP dependent RNA helicase DDX3X inhibitors, reverse transcriptase priming complex inhibitors, HIV gene therapy, PI3K inhibitors, compounds, such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US 20140221378 (Japan Tobacco), WO 2010/130034 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO 2013/159064 (Gilead Sciences) and WO 2012/003498 (Gilead Sciences) and WO 2013/006792 (Pharma Resources), and other drugs for treating HIV, and combinations thereof.

In certain embodiments, the additional therapeutic chosen from: HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, bictegravir and lamivudine are formulated as a tablet that may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacoki-netic enhancers, and combinations thereof. In certain embodiments, such tablets are suitable for once daily dosing.

In certain embodiments, the additional therapeutic agent may be chosen from one or more of:

(1) Combination drugs chosen from: ATRIPLA® (efavirenz+tenofovir disoproxil fumarate+emtricitabine), COMPLERA® (EVIPLERA®, lamivudine+tenofovir disoproxil fumarate+emtricitabine), STRIBILD® (elvitegravir+cobicistat+tenofovir disoproxil fumarate+emtricitabine), lamivudine+nevirapine+zidovudine, atazanavir sulfate+cobicistat, darunavir+cobicistat, efavirenz+lamivudine+tenofovir disoproxil fumarate, Vacc-4x+romidepsin, APH-0812, raltegravir+lamivudine, KALETRA® (ALUVIA®, lopinavir+ritonavir), atazanavir sulfate+ritonavir, COMBIVIR® (zidovudine+lamivudine, AZT+3TC), EPZICOM® (Kivexa , abacavir sulfate+lamivudine, ABC+3TC), TRIZIVIR® (abacavir sulfate+zidovudine+lamivudine, ABC+AZT+3TC), TRUVADA® (tenofovir disoproxil fumarate+emtricitabine, TDF+FTC), tenofovir+lamivudine, atazanavir+cobicistat, doravirine+lamivudine+tenofovir disoproxil fumarate, doravirine+lamivudine+tenofovir disoproxil and lamivudine+tenofovir disoproxil fumarate; (2) HIV protease inhibitors chosen from : amprenavir, atazanavir, fosamprenavir, fosam-prenavir calcium, indinavir, indinavir sulfate, lopinavir, ritonavir, nelfinavir, nelfinavir mesylate, saquinavir, saquinavir mesylate, tipranavir, brecanavir, darunavir, DG-17, TMB-657 (PPL-100), TMC-310911, and TMB-657;

(3) HIV non-nucleoside or non-nucleotide inhibitors of reverse transcriptase chosen from: rilpivirine, delavirdine, delavirdine mesylate, nevirapine, (+),etravirine, dapivirine, doravirine, efavirenz, KM023, VM-1500, lentinan, AIC-292, EFdA (4′-Ethynyl-2-Fluoro-2′-Deoxyadenosine, or otherwise known as MK-8591) and KM-023;

(4) HIV nucleoside or nucleotide inhibitors of reverse transcriptase chos en from: VIDEX® and VIDEX® EC (didanosine, ddl), zidovudine, emtricitabine, didanosine, stavudine, zalcitabine, censavudine, abacavir, abacavir sulfate, amdoxovir, elvucitabine, alovudine, phosphazid, fozivudine tidoxil, apricitabine, amdoxovir, KP-1461, fosalvudine tidoxil, tenofovir, tenofovir disoproxil, tenofovir disoproxil fumarate, tenofovir disoproxil hemifumarate, adefovir, adefovir dipivoxil, and festinavir.

(5) HIV integrase inhibitors chosen from: curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phen-ethyl ester, derivatives of caffeic acid phenethyl ester, tyrphostin, derivatives of tyrphostin, quercetin, derivatives of quercetin, raltegravir, elvitegravir, cab oteg rav ir, and TIVICAY® (dolutegravir);

(6) HIV non-catalytic site, or allosteric, integrase inhibitors (NCINI) chosen from: CX-05168, CX-05045 and CX-14442;

(7) HIV gp41 inhibitors chosen from: enfuvirtide, sifuvirtide and albuvirtide;

(8) HIV entry inhibitors, such as cenicriviroc;

(9) HIV gp120 inhibitors chosen from: Radha-108 (Receptol) and BMS-663068;

(10) CCRS inhibitors chosen from: aplaviroc, vicriviroc, maraviroc, cenicriviroc, PRO-140, Adaptavir (RAP-101), nifeviroc (TD-0232), TD-0680, TBR-220 (TAK-220) and vMIP (Haimipu);

(11) CD4 attachment inhibitors, such as ibalizumab;

(12) CXCR4 inhibitors chosen from: plerixafor, ALT-1188, vMIP and Haimipu;

(13) Pharmacokinetic enhancers chosen from: cobicistat and ritonavir;

(14) Immune-based therapies chosen from: dermaVir, interleukin-7, lexgenleucel-T (VRX-496), plaquenil (hydroxychloroquine), proleukin (aldesleukin, IL-2), interferon alfa, interferonalfa-2b, interferon alfa-n3, pegylated interferon alfa, interferon gamma, hydroxyurea, mycophenolate mofetil (MPA) and its ester derivative mycophenolate mofetil (MMF), WF-10, ribavirin, IL-2, IL-2 XL, IL-12, polymer polyethyleneimine (PEI), Gepon, VGV-1, MOR-22, toll-like receptors modulators (tlr1, tlr2, tlr3, tlr4, tlr5, tlr6, tlr7, tlr8, tlr9, tlr1O, tlr11, tlr12 and tlr13), BMS-936559, rintatolimod and IR-103; (15) HIV vaccines chosen from: peptide vaccines, recombinant subunit protein vaccines, live vector vaccines, DNA vaccines, virus-like particle vaccines (pseudovirion vaccine), CD4-derived peptide vaccines, vaccine combinations, rgp120 (AIDSVAX), ALVAC HIV (vCP1521)/AIDSVAX B/E (gp120) (RV144), Remune, ITV-1, Cantre Vir,Ad5-ENVA-48, DCVax-001 (CDX-2401), PEP-6409, Vacc-4x, Vacc-C5, VAC-3S, multiclade DNA recombinant adenovirus-5 (rAdS), Pennvax-G, YRC-HIV MAB060-00-AB, AVX-101, Tat Oyi vaccine, AVX-201, HIV-LAMP-vax, Ad35, Ad35-GRIN, NAcGM3NSSP ISA-51, poly-ICLC adjuvanted vaccines, Tatlmmune, GTU-multi-HIV (FIT-06), AGS-004, gp140[delta]V2.TVI+MF-59, rVSVIN HIV-1 gag vaccine, SeV-Gag vaccine, AT-20, DNK-4, Ad35-GRIN/ENV, TBC-M4, HIVAX, HIVAX-2, NYVAC-HIV-PT1, NYVAC-HIV-PT4, DNA-HIV-PT123, VIChREPOL®, rAAV1-PG9DP, GOVX-B11, GOVX-B21, ThV-01, TUTI-16, VGX-3300, TVI-HIV-1, Ad-4 (Ad4-env Clade C+Ad4-mGag), EN41-FPA2, PreVaxTat, TL-01, SAV-001, AE-H, MYM-VIOI, CombiHIVvac, ADVAX, MYM-V201, monomeric gp120 HIV-1 subtype C vaccine (Novartis), MVA-CMDR, MVATG-17401, ETV-01, CDX-1401, rcAd26.MOS1.HIV-Env, and DNA-Ad5 gag/pol/nef/nev (HVTN505);

(16) HIV antibodies, bispecific antibodies and “antibody-like” therapeutic proteins (such as DARTs®, Duo-bodies®, Bites®, XmAbs®, TandAbs®, Fab derivatives), including BMS-936559, TMB-360 and those targeting HIV gp120 or gp41 are chosen from: bavituximab, UB-421, C2F5, C2G12, C4E10, C2F5+C2G12+C4E10, 3-BNC-117, KD-247, PGT145, PGT121, MDXO1O (ipilimumab), VRCO1, A32, 7B2, 10E8, VRC-07-523 and VRC07;

(17) latency reversing agents chosen from: histone deacetylase inhibitors such as Romidepsin, vorinostat, panobinostat; proteasome inhibitors such as VELCADE®; protein kinase C (PKC) activators such as Indolactam, prostratin, ingenol B and DAG-lactones, lono-mycin, GSK-343, PMA, SAHA, BRD4 inhibitors, IL-15, JQ1, amphotericin B, and disulfram;

(18) HIV nucleocapsid p7 (NCp7) inhibitors, such as azodicarbonamide;

(19) HIV maturation inhibitors chosen from: BMS-955176 and GSK-2838232;

(20) P13K inhibitors chosen from: idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401;

(21) the compounds disclosed in WO 2004/096286 (Gilead Sciences), WO 2006/110157(Gilead Sciences), WO 2006/015261 (Gilead Sciences), WO 2013/006738 (Gilead Sciences), US 2013/0165489 (University of Pennsylvania), US20140221380 (Japan Tobacco), US20140221378 (Japan Tobacco), WO 2013/006792 (Pharma Resources), WO 2009/062285 (Boehringer Ingelheim), WO 2010/130034 (Boehringer Ingelheim), WO 2013/091096A1 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences); and (22) other drugs for treating HIV chosen from: REP 9, cytolin, CYT-107, alisporivir, BanLec, MK-8507, AG-1105, TR-452, MK-8591, REP 9, NOV-205, IND-02, metenkefalin, PGN-007, Acemannan, Gamimune, SCY-635, prolastin, 1,5-dicaffeoylquinic acid, BIT-225, RPI-MN, VSSP, Hlviral, IM0-3100, SB-728-T, RPI-MN, VIR-576, HGTV-43, MK-1376, rHIV7-shl-TAR-CCR5RZ, MazF gene therapy, BlockAide, ABX-464, SCY-635, naltrexone, AAV-eCD4-Ig gene therapy, TEV-90110, TEV-90112, deferiprone, and PA-1050040 (PA-040).

In certain embodiments, bictegravir and lamivudine or a pharmaceutical composition thereof, are combined with one, two, three, four or more additional therapeutic agents. The one, two, three, four or more additional therapeutic agents can be different therapeutic agents selected from the same class of therapeutic agents, and/or they can be selected from different classes of therapeutic agents.

In a particular embodiment, bictegravir and lamivudine or a pharmaceutical composition thereof is combined with one, two, three, four or more additional therapeutic agents selected from raltegravir, Truvada® (tenofovir disoproxil fumarate+emtricitabine, TDF+FTC), maraviroc, enfuvirtide, EPZICOM® (KIVEXA®, abacavir sulfate+lamivu-dine, ABC+3TC), TRIZIVIR® (abacavir sulfate+zidovudine+lamivudine, ABC+AZT+3TC), adefovir, adefovir dipivoxil, STRIBILD (⁴) (elvitegravir+cobicistat+tenofovir disoproxil fumarate+emtricitabine), raltegravir+lamivudine, COMPLERA® (EVIPLERA®, lamivudine+tenofovir disoproxil fumarate+emtricitabine), Cobicistat, ATRIPLA® (efavirenz+tenofovir disoproxil fumarate+emtricitabine), atazanavir sulfate+cobicistat, atazanavir+cobicistat, darunavir+cobicistat, atazanavir, atazanavir sulfate, elvitegravir, ALUVIA® (KALETRA®, lopinavir+ritonavir), ritonavir, emtricitabine, atazanavir sulfate+ritonavir, darunavir, lamivudine, Prolastin, fosamprenavir, fosamprenavir calcium, efavirenz, COMBIVIR® (zidovudine+lamivudine, AZT+3TC), etravirine, nelfinavir, nelfininavir mesylate, interferon, didanosine, stavudine, indinavir, indinavir sulfate, tenofovir+lamivudine, zidovudine, nevirapine, saquinavir, saquinavir mesylate, aldesleukin, zalcitabine, tipranavir, amprenavir, delavirdine, delavirdine mesylate, Radha-108 (Receptol), Rivira, lamivudine+tenofovir diso-proxil fumarate, efavirenz+lamivudine+tenofovir disoproxil fumarate, phosphazid, lamivudine+nevirapine+zidovudine, abacavir, abacavir sulfate, tenofovir, tenofovir disoproxil and tenofovir disoproxil fumarate.

In one embodiment, bictegravir and lamivudine are administered with emtricitabine (in the first case). In certain embodiments, when bictegravir and lamivudine or a pharmaceutical composition thereof is combined with one or more additional therapeutic agents as described above, the components of the composition are administered as a simultaneous or sequential regimen. When administered sequentially, either combination may be administered in two or more administrations.

In certain embodiments, bictegravir and lamivudine or a pharmaceutical composition thereof are combined with one or more additional therapeutic agents, in a unitary dosage form for simultaneous administration to a patient, for example as a solid dosage form for oral administration. In certain embodiments, bictegravir and lamivudine or a pharmaceutical composition thereof are administered with one or more additional therapeutic agents. Co-administration of bictegravir and lamivudine or a pharmaceutical composition thereof with one or more additional therapeutic agents generally refers to simultaneous or sequential administration of a compound disclosed herein and one or more additional therapeutic agents, such that therapeutically effective amounts of the compound disclosed herein and one or more additional therapeutic agents are both present in the body of the patient.

Co-administration includes administration of unit dosages of bictegravir and lamivudine or a pharmaceutical composition thereof before or after administration of unit dosages of one or more additional therapeutic agents, for example, administration of bictegravir and lamivudine or a pharmaceutical composition thereof, within seconds, minutes, or hours of the administration of one or more additional therapeutic agents. For example, in some embodiments, a unit dose of bictegravir and lamivudine or a pharmaceutical composition thereof is administered first followed within seconds or minutes by administration of a unit dose of one or more additional therapeutic agents. Alternatively, in other embodiments, a unit dose of one or more additional therapeutic agents is administered first, followed by administration of a unit dose of bictegravir and lamivudine or a pharmaceutical composition thereof within seconds or minutes. In some embodiments, a unit dose of bictegravir and lamivudine or a pharmaceutical composition thereof, is administered first, followed, after aperiod of hours (e.g., 1-12 hours), by administration of a unit dose of one or more additional therapeutic agents. In other embodiments, a unit dose of one or more additional therapeutic agents is administered first, followed, after aperiod of hours (e.g., 1-12 hours), by administration of a unit dose of bictegravir and lamivudine or a pharmaceutical composition thereof.

In another embodiment, the combination of bictegravir and lamivudine is administered to the patient once a day.

In another embodiment, the combination of bictegravir and lamivudine is administered to the patient twice a day.

In another embodiment, bictegravir is administered to the patient at about 5 mg, about 10 mg, about 25 mg, about 50 mg, about 75 mg, or about 100 mg, once, twice or three times a day.

In another embodiment, bictegravir is administered to the patient at about 5 mg to 100 mg, at about 25 mg to 75 mg, at about 35 mg to 65 mg or about 45 mg to 55 mg, once or twice per day.

In another embodiment, lamivudine is administered to the patient as a tablet, wherein the effective amount is between 1 to 1000 mg of active ingredient per unit dosage form; or between 100mg and 300 mg of active ingredient per unit dosage form. In another embodiment, lamivudine is administered to the patient in a tablet form at about 100mg, 150mg or 300mg, once per day or twice a day.

In another embodiment, a single dosage form containing bictegravir and lamivudine contains about 50 or 75 mg of bictegravir and 100, 150 or 300 mg of lamivudine.

In another embodiment, this invention relates to a combination comprising bictegravir or a pharmaceutically acceptable salt thereof and lamivudine or a pharmaceutically acceptable salt thereof.

In another embodiment, this invention relates to the above combinations for use in medical therapy. In a further embodiment, the above combinations are for use in a method for treating HIV infection. In one embodiment, the targeted population for treatment of HIV infection are human individuals who are virologically suppressed (HIV-1 RNA<50c/mL). In one embodiment, the above combinations are for use in a method of maintaining HIV-1 RNA<50c/mL. In a further embodiment, HIV-1 RNA <50c/mL is maintained at 48 weeks.

In another embodiment, this invention relates to pharmaceutical composition comprising a combination comprising bictegravir or a pharmaceutically acceptable salt thereof and lamivudine or a pharmaceutically acceptable salt thereof.

In another embodiment, this invention relates to pharmaceutical composition comprising a combination comprising bictegravir or a pharmaceutically acceptable salt thereof and lamivudine or a pharmaceutically acceptable salt thereof in association with one or more pharmaceutically acceptable carriers therefor.

In another embodiment, the present invention provides for the use of bictegravir and lamivudine for the manufacture of a medicament for the treatment of treating or preventing HIV.

### DETAILED DESCRIPTION OF THE INVENTION

Definitions

As used herein, the term “co-administer” refers to administration of two or more agents within a 24-hour period of each other, for example, as part of a clinical treatment regimen. In other embodiments, “co-administer” refers to administration of two or more agents within 2 hours of each other. In other embodiments, “co-administer” refers to administration of two or more agents within 30 minutes of each other. In other embodiments, “co-administer” refers to administration of two or more agents within 15 minutes of each other. In other embodiments, “co-administer” refers to administration at the same time, either as part of a single formulation or as multiple formulations that are administered by the same or different routes.

“Therapeutically effective amount” or “effective amount” refers to that amount of the compound being administered that will prevent a condition, or will relieve to some extent one or more of the symptoms of the disorder being treated. Pharmaceutical compositions suitable for use herein include compositions wherein the active ingredients are contained in an amount sufficient to achieve the intended purpose. Determination of a therapeutically effective amount is well within the capability of those skilled in the art, especially in light of the detailed disclosure provided herein.

As used herein, “treatment”, refers to inhibition, reduction, elimination or alleviation of a disease, as well as prevention.

HIV mutations showing NRTI resistance is well documented. Examples of HIV mutations which show resistance to TAF (TAF has the same resistance profile tenovofir and tenofovir disoproxil) and FTC are published, such as, in Characterization of HIV-1 Resistance to Tenofovir Alafenamide In vitro, Antimicrobial Agents and Chemotherapy, vN. A. Margot et al., Volume 59 Number 10 (2015). Also is published online at https://hivdb.stanford.edu/dr-summary/resistancc-notes/NRTI/.

### Combinations and Methods of Treatment

A method for the treatment or prophylaxis of diseases, disorders, and conditions is provided herein. An example of a disease, disorder, or condition includes, but is not limited to, a retrovirus infection, or a disease, disorder, or condition associated with a retrovirus infection. Retroviruses are RNA viruses and are generally classified into the alpharetrovirus, betaretrovirus, deltaretrovirus, epsilonretrovirus, gammaretrovirus, lentivirus, and spumavirus families. Examples of retroviruses include, but are not limited to, human immunodeficiency virus (HIV).

The active agents of the disclosed combination therapy may be administered to a human in any conventional manner. While it is possible for the active agents to be administered as compounds, in one embodiment of the invention, they are administered as a pharmaceutical composition that can include contact with an acid or base, either in an ionic salt form or in contact with the base or acid (i.e., co-formers) without sharing ions. The salt, acid or base co-former, carrier, or diluent should be acceptable, in the sense of being compatible with the other ingredients and not deleterious to the recipient thereof. Examples of carriers or diluents for oral administration include, but are not limited to: cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), sodium lauryl sulfate, mannitol, sodium stearyl fumarate, and talc. Examples of salts and acid or base co-formers include fumarate, hemifumarate, sodium, and hydrochloride.

The pharmaceutical compositions may be prepared by any suitable method, such as those methods well known in the art of pharmacy, for example, methods such as those described in Gennaro, et al., REMINGTON′S PHARMACEUTICAL SCIENCES (18th ed., Mack Publishing Co., 1990), especially “Part 8: Pharmaceutical Preparations and their Manufacture”. Such methods include the step of bringing into association the compounds with the carrier or diluents and, optionally, one or more accessory ingredients. Such accessory ingredients include, but are not limited to: fillers, binders, excipients, disintegrants, lubricants, colorants, flavoring agents, sweeteners, preservatives (e.g., antimicrobial preservatives), suspending agents, thickening agents, emulsifying agents, and/or wetting agents.

In practice, the amount of each compound to be administered ranges from about 0.001 to 100 mg per kg of body weight, such total dose being given at one time or in divided doses. Each compound will be administered as a formulation in association with one or more pharmaceutically acceptable excipients. Alternatively, both compounds will be combined and administered as a formulation in association with one or more pharmaceutically acceptable excipients. The choice of excipient will, to a large extent, depend up on factors such as the particular mode of administration, the effect of the excipient on solubility and stability, and the nature of the dosage form. Such compositions and methods for their preparation may be found, for example, in REMINGTON′S PHARMACEUTICAL SCIENCES (19th Edition, Mack Publishing Company, 1995).

In the following description of the examples, specific embodiments in which the invention may be practiced are described. These embodiments are described in sufficient detail to enable those skilled in the art to practice the invention. Other embodiments may be utilized and logical and other changes may be made without departing from the scope of the invention. The following detailed description is, therefore, not to be taken in a limiting sense, and the scope of the invention is defined only by the appended claims, along with the full scope of equivalents to which such claims are entitled.

### EXAMPLES

Example 1: HIV Cell Line Assay. The HIV cell line experiments taught by Kobayashi, et al., Antimicrobial Agents and Chemotherapy, 55: 814-815 (2011) are followed to test the antiviral abilities of two disclosed and claimed combination of lamivudine and bictegravir as compared with the antiviral abilities of each of these compounds alone.

Cells and viruses. Cells of MT-4, a human T-cell leukemia virus type I (HTLV-1)-transformed human T-cell line, are maintained as described previously [12]. 293T cells are maintained in Duibecco's modified Eagle medium (DMEM)-F-12 medium containing 10% fetal bovine serum (FBS). Peripheral blood mononuclear cells (PBMCs) are derived from whole blood samples obtained from HIV-negative donors. PBMCs are separated from whole blood by density gradient centrifugation with Ficoll-Paque Plus®(GE Healthcar®, Waukesha, Wis) according to the manufacturer's instructions and are stimulated by the addition of either 20 U/ml of interleukin-2 (IL-2) or 10% natural T-cell growth factor (ZeptoMetrix®, Buffalo, N.Y.) plus 5 to 10 μg/ml of phytohemagglutinin (PHA). Molt-4 cells persistently infected with HIV-IIIB and MT-2 cells [16] are obtained from S. Harada (Kumamoto University, Kumamoto, Japan). HeLa-CD4 cells containing an HIV-1 long terminal repeat. (LTR)-driven β-galactosidase reporter gene have been described previously [20]. MAGI-CCR5 cells have been described previously [9]). HIV-1 strain IIIB is derived from cell-free supernatants of cultures of the chronically infected cell line, H93B (H9/HILV-IIIB). HIV-1 strain Ba-L is purchased from Advanced Biotechnologies Inc. ® (Columbia, Md.) and is expanded in PHA-activated PBMCs, while HIV-1 NL432 [1] is obtained from A. Adachi (Tokushima University, Tokushima, Japan). Plasmid pGJ3-Luci, containing a replication-defective HIV lentiviral vector expressing luciferase [21], is licensed from Christian Jassoy (University of Leipzig), and is used to create stocks of a vesicular stomatitis virus glycoprotein G (VSV-G)-pseudotyped self-inactivating pseudo-HIV (PHIV) lentiviral vector by cotransfection, along with plasmid PVSV-G (Ciontech®) into CIP4 cells (a derivative of the 293T human renal epithelial cell line that expresses macrophage scavenger receptor SRA-I to improve adherence to plastic) and harvesting of the cell-free supernatant.

Antiviral assay in MT-4 cells. MT-4 cells growing exponentially at a density of 5×1.0⁵ or 6×10⁵/ml are infected with HIV-1 strain IIIB at a viral multiplicity of infection of 0.001 or a 50% tissue culture infective dose of 4 to 10. The cells are then aliquoted to 96-well plates in the presence of varying concentrations of compounds. After incubation for 4 or 5 days, antiviral activity is determined by a cell viability assay that either measures bioluminescence with a CellTiter-Glo® luminescent reagent (Promega Corporation®, Madison, Wis.) or measured absorbance at 560 and 690 nm using the yellow tetrazolium MIT reagent [3-(4,5-dimethyl-2-thiazolyl)-2,5-diphenyltetrazoli um bromide].

Pseudo-HIV assay. The antiviral activities of bictegravir and lamivudine alone, as well as the combination of bictegravir and lamivudine are measured in a single-round assay using a self-inactivating PHIV lentiviral vector. CIP4 cells (2×10⁴/well) infected with an amount of PHIV sufficient to produce approximately 50,000 relative light units are added to 96-well black, clear-bottom plates and were incubated for 2 days with all three compounds at varying concentrations. Infected cells are measured as a function of luciferase activity in a luminometer using the Steady-Glo® reagent (Promega Corporation®).

Antiviral assay in PBMCs. In one 96-well culture plate, PHA- and IL-2-stimulated PBMCs (4×10⁵/well) are pre-incubated with each compound alone, and then for each of the two above combinations of the compounds, for 1 h, while HIV-1 strain, Ba-L, is mixed with the same compound in a second plate. An aliquot of the Ba-L-compound mixture is then transferred to the PBMC-compound mixture and is incubated for 7 days. After this incubation, supernatants are assayed for reverse transcriptase (RT) activity by incorporation of [methyl-³H] dTTP to measure viral replication, as previously described [15].

Effects of human serum and serum proteins. The effect of the presence of human serum albumin (HSA; 20 or 40 mg/ml), β₁-glycoprotein (AAG; 2 mg/ml), or human serum (HS; up to 30% or 50% is used, and results were extrapolated up to 100%) on the antiviral activity of each of bictegravir and lamivudine alone is evaluated in the PHIV and MT-4 assay systems. To estimate the effects of the fold shift in protein binding, antiviral activity is tested with the addition of various concentrations of human serum to the HIV-1 IIIB replication assay mixture in MT-4 cells, as previously described [15]. The protein-adjusted half-maximal effective concentration (PA-EC₅₀) is estimated by multiplying the EC₅₀ in PBMCs by the fold shift value. The same experiment is conducted using the combination of bictegravir plus lamivudine.

Combination antiviral activity assay in MT-4 cells. The in vitro combination activity relationships of (1) bictegravir alone; (2) lamivudine alone; and (3) bictegravir and lamivudine are determined as previously described [39]. Multiple concentrations of the compounds are tested in checkerboard dilution fashion in the presence and absence of dilutions of approved anti-HIV drugs, adethvir, or ribavirin. The assay used HIV-1 IIIB-infected MT-4 cells, and the interaction of compound combinations is analyzed by dose wise additivity-based calculations to quantify deviation from dose wise additivity at the 50% level. Wells containing the top concentration of compounds by themselves are compared to wells with the top concentration of each of the two compound combinations in order to show that combination effects are due to the drugs used, and not simply to toxicity. Assays with the MT-4 system format are run as described previously [15]. Fractional inhibitory concentration (FIC) values in the range of 0.1 to 0.2 indicate weak synergy; values that approach 0.5 indicate strong synergy; and positive values of 0.1 to 0.2 indicate weak antagonism. The effects of the anti-hepatitis B virus (anti-HBV) and anti-HCV agents adefovir and ribavirin on; (1) bictegravir alone; (2) lamivudine alone; (3) bictegravir and lamivudine are examined using linear regression, as described previously [41]. Because the HIV-1 IIIB MT-4 system is CXCR4-based, the CCR5 inhibitor, maraviroc, is evaluated in a checker-board dilution format using MAGI-CCR5 cells with the Gal Screen reagent (Tropix®, Bedford, Mass.) for chemiluminescent endpoints, and data are analyzed as described by Prichard and Shipman [37]by using the MacSynergy II® program. Synergy volumes in the range of −50 to 50 define additivity; <−50, antagonism; and >50, synergy.

The results of these experiments are expected to show that the combination bictegravir and lamivudine is synergistic in their antiviral abilities as compared to the antiviral abilities of each compound alone. Specifically, in each of the HIV cell-based assays described above, both these combinations are expected to be synergistic in each combination's ability to inhibit HIV replication in cells over that expected with each of the three compounds alone.

### REFERENCES¹

The reference numbering used in this example is the same as that used in Kobayashi, et al., supra.

¹1. Adachi, et al., J. Virol. 59:284 -291 (1986).

9. Chackerian, et al., J Virol. 71:3932-3939 (1997).

12. Daluge, et al., Antimicrob. Agents Chemother. 38:1590-1603. (1994).

15. Garvey, et al., Antimicrob. Agents Chemother. 52:901-908 (2008).

20. Isaka, et al., Virology 264:237-243 (1999).

21. Jármy, et Med. Virol. 64:223-231 (2001).

37. Prichard, et al., Antiviral Res. 14:181-205 (1990).

39. Selleseth, et c.d., Antinherob. Agents Chemother. 47:1468-1471 (2003).

41. Tukey, et al., Biometrics 41:295-301 (1985).

What is claimed is:

1. A method for treating or preventing human immunodeficiency virus (HIV) in a patient in need thereof, comprising administering to the patient a pharmaceutically effective amount of a compound of Formula I: 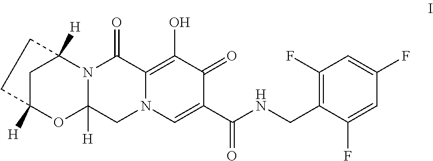 [SMILES: O=C(NCc1c(F)cc(F)cc1F)c1cn2c(c(O)c1=O)C(=O)N1C(C2)O[C@@H]2CC[C@H]1C2]
or a pharmaceutical composition thereof; and
a pharmaceutically effective amount of a compound of Formula II: 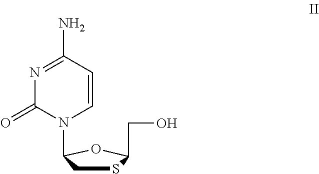
or a pharmaceutical composition thereof.

2. The method of claim 1, wherein the compound of Formula I and the compound of Formula II are co-administered in separate dosage forms.

3. The method of claim 1, wherein the compound of Formula I and the compound of Formula II are co-administered in a single dosage form.

4. A pharmaceutical composition comprising a compound of Formula I: 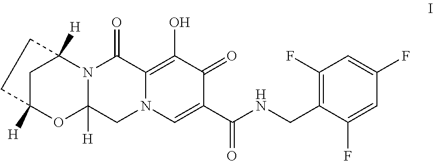 [SMILES: O=C(NCc1c(F)cc(F)cc1F)c1cn2c(c(O)c1=O)C(=O)N1C(C2)O[C@@H]2CC[C@H]1C2]
and a compound of Formula II: 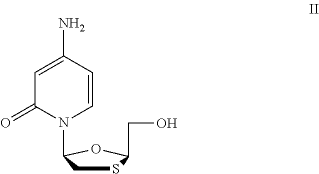
and at least one pharmaceutically acceptable carrier.

5. The pharmaceutical composition of claim 4 containing 75 mg of Formula I compound and 300 mg of Formula II compound.

6. A kit comprising:
(1) a composition comprising bictegravir; and
(2) a composition comprising lamivudine; and
(3) instructions for their co-administration.

7. A kit comprising:
a composition comprising bictegravir and lamivudine for their co-administration.

8. A combination comprising a compound of Formula I: 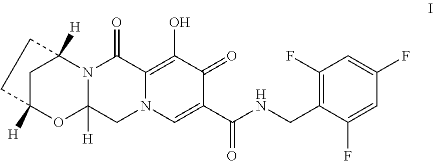 [SMILES: O=C(NCc1c(F)cc(F)cc1F)c1cn2c(c(O)c1=O)C(=O)N1C(C2)O[C@@H]2CC[C@H]1C2]
or a pharmaceutically acceptable salt thereof; and
a compound of Formula II: 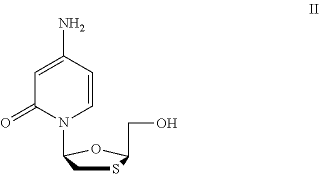
or a pharmaceutically acceptable salt thereof.

9. A pharmaceutical composition comprising the combination of claim 8 in association with one or more pharmaceutically acceptable carriers therefor.

10.-11.(canceled)
